(19) United States
(12) Patent Application Publication
(10) Pub. No.: US 2025/0073076 A1
(43) Pub. Date: Mar. 6, 2025

(54) OPERATING ENVIRONMENT WITH INTEGRATED DIAGNOSTIC AND TREATMENT EQUIPMENT

(71) Applicant: Alcon Inc., Fribourg (CH)

(72) Inventors: Patrick TERRY, Goleta, CA (US); Mark SMYTHE, Fort Worth, TX (US); Sissimos LEMONIS, Rückersdorf (DE); Mark Andrew ZIELKE, Lake Forest, CA (US); Lance NOLLER, Lake Forest, CA (US); Nanhong LOU, Southlake, TX (US)

(21) Appl. No.: 18/816,873

(22) Filed: Aug. 27, 2024

Related U.S. Application Data
(60) Provisional application No. 63/579,283, filed on Aug. 28, 2023.

(51) Int. Cl. A61F 9/008; A61B 3/103; A61B 34/30; A61F 9/00

(52) U.S. Cl. CPC A61F 9/00804; A61F 9/00827; A61F 9/00836; A61B 3/103; A61B 34/30; A61F 2009/0035; A61F 2009/00851; A61F 2009/00853; A61F 2009/00872

(57) ABSTRACT

A system includes an actuator, one or more items of diagnostic equipment configured to perform ophthalmic measurement, and one or more items of treatment equipment configured to facilitate performance of an ophthalmic treatment with respect to an eye of a patient. A controller is coupled to the actuator and is configured to cause the actuator to transfer the one or more items of diagnostic equipment and the one or more items of treatment equipment into and out of a region in front of an eye of the patient. The ophthalmic treatment may be a LASIK or SMILE treatment using refractive error and/or eye geometry measured using the diagnostic equipment.

### CROSS-REFERENCE TO RELATED APPLICATIONS

This application claims priority to U.S. Provisional Application No. 63/579,283, filed on Aug. 28, 2023, which is hereby incorporated by reference in its entirety.

### BACKGROUND

The present disclosure relates generally to performing ophthalmic surgery.

Light received by the eye is focused by the cornea and lens of the eye onto the retina at the back of the eye, which includes the light sensitive cells. Ophthalmic treatments such as laser-assisted in-situ keratomileusis (LASIK) and small incision lenticular extraction (SMILE) are performed on the cornea to correct the refractive error of the eye.

It would be an advancement in the art to facilitate the performance of LASIK and SMILE treatments.

### SUMMARY

In certain embodiments, a system for performing ophthalmic surgery includes an actuator, one or more items of diagnostic equipment configured to perform ophthalmic measurement, and one or more items of treatment equipment configured to facilitate performance of an ophthalmic treatment with respect to an eye of a patient. A controller is coupled to the actuator and is configured to cause the actuator to transfer the one or more items of diagnostic equipment and the one or more items of treatment equipment into and out of a region in front of an eye of the patient.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is schematic diagram of an operating environment including robotically actuated diagnostic and treatment equipment in accordance with certain embodiments.

FIG. 1B is a diagram illustrating an operating environment including shared diagnostic and treatment equipment in accordance with certain embodiments.

FIG. 1C is a diagram illustrating the operating environment of FIG. 1B with the diagnostic and treatment equipment in different patient areas in accordance with certain embodiments.

FIG. 1D is a diagram illustrating an operating environment including diagnostic and treatment equipment and an actuated instruments in accordance with certain embodiments.

FIG. 2A is a process flow diagram of a method for performing a LASIK treatment in accordance with certain embodiments.

FIG. 2B is a process flow diagram of a method for performing intraoperative diagnostic measurements in accordance with certain embodiments.

FIG. 3A is a cross-sectional view illustrating a SMILE treatment.

FIG. 3B is a top view illustrating a SMILE treatment.

FIG. 4A is a process flow diagram of a method for performing a SMILE treatment in accordance with certain embodiments.

FIG. 4B is a process flow diagram of a method for performing a SMILE treatment with an additively manufactured lenticle in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

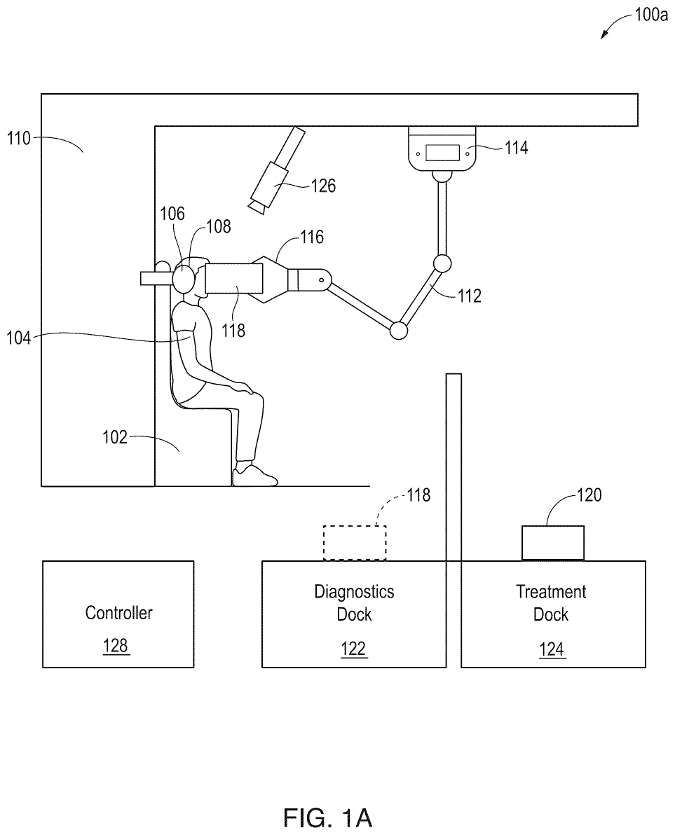

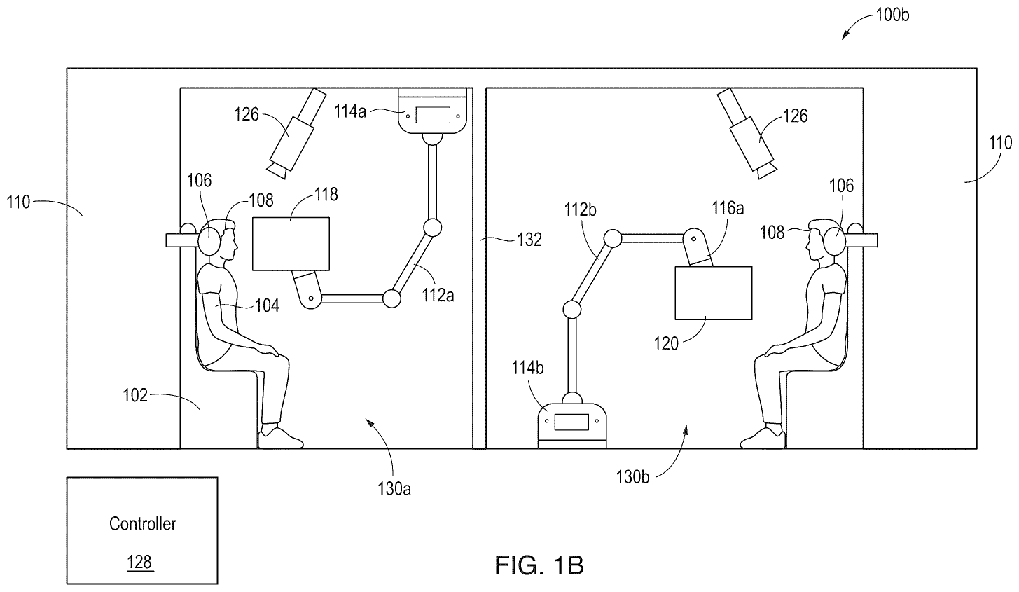

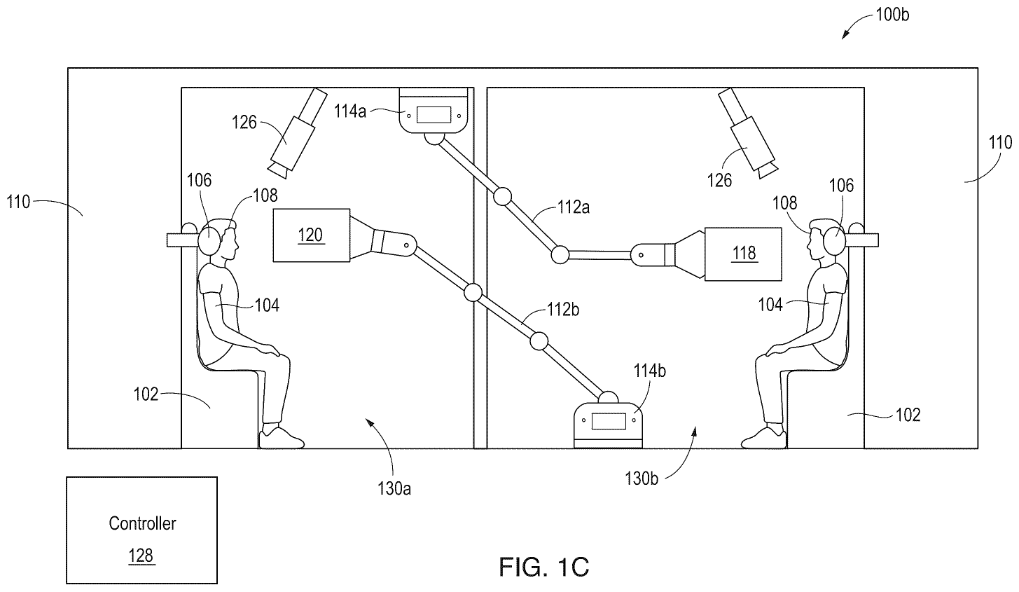

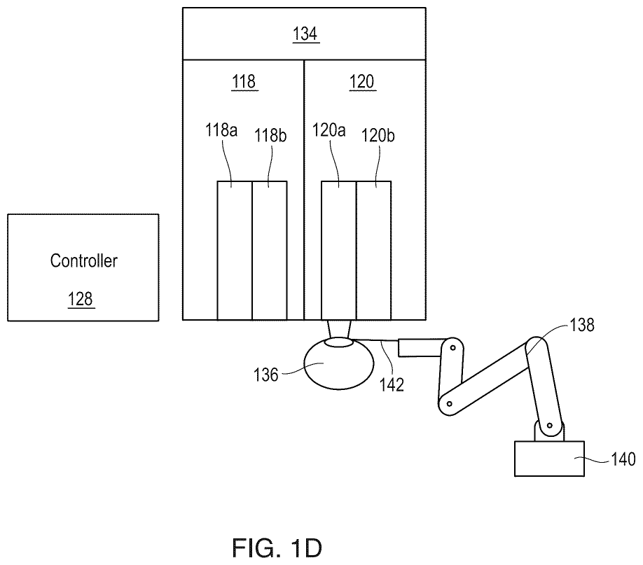

Referring to FIG. 1A, an operating environment 100a includes a patient support 102 on which a patient 104 will rest when undergoing an ophthalmic treatment. The patient support 102 may support the patient 104 in an upright, leaning, seated, or supine position. A clamp 106, or other type of restraint, is positioned on or near the patient support 102 and is configured to reduce motion of the patient's head 108 when undergoing the ophthalmic treatment.

The patient support 102 and clamp 106 may be fixed relative to one another, such as by mounting to a common support structure 110. The support structure 110 may be mounted to a floor or wall or may be sufficiently massive to effectively prevent movement during use.

A robotic arm 112 is mounted to a base 114 that is fixed relative to the clamp 106, such as by being mounted to a floor, wall, or ceiling of the same structure or mounting to the support structure 110 The robotic arm 112 may be embodied as any arrangement of actuators providing five, six, or more degrees of freedom. The robotic arm 112 may be implemented as a serial robotic arm or other type of robotic arm.

An end effector 116 is moved by the robotic arm 112 throughout the degrees of freedom of the robotic arm 112. The end effector 116 is configured to grasp or otherwise selectively secure to and release from diagnostic equipment 118 and treatment equipment 120. The diagnostic equipment 118 includes one or more items of equipment used to visualize or measure an eye of the patient. The diagnostic equipment 118 may include one or more of an optical coherence tomography (OCT) imaging device, wavefront analyzer, surgical microscope (mono- or stereoscopic), autorefractor, scanning laser ophthalmoscope (SLO), multi-spectral imaging (MSI) or hyperspectral imaging (HSI) camera, fundus autofluorescence (FAF) imaging device, or other type of imaging device. The diagnostic equipment 118 may include a sensor such as an intraocular pressure (IOP) sensor, or other type of sensor.

The end effector 116 is further configured to grasp or otherwise selectively secure to and release from treatment equipment 120. The treatment equipment 120 is configured to perform or assist in the performance of an ophthalmic treatment, such as LASIK, SMILE, photorefractive keratectomy (PRK), phacoemulsification, intraocular lens (IOL) placement, implantable contact lens (ICL) placement, glaucoma surgery (e.g., minimally invasive glaucoma surgery (MIGS)), vitrectomy, retinal attachment, or other ophthalmic treatment. In the case of LASIK or SMILE, the treatment equipment 120 may include a laser and optics for directing and focusing the laser for creating a flap and performing ablation in the case of LASIK or creating a lenticle in the case of SMILE.

When not in use, the diagnostic equipment 118 may be placed on or in a diagnostic dock 122. When not in use, the treatment equipment may be placed on or in a treatment dock 124. The diagnostic dock 122 and treatment dock 124 may include structures for securing to the diagnostic equipment 118 and treatment equipment 120, respectively. The diagnostic dock 122 and treatment dock 124 may include structures for sanitizing the diagnostic equipment 118 and treatment equipment 120, respectively, for use with a next patient or the next eye of the same patient 104. The diagnostic dock 122 and treatment dock 124 may include structures for resupplying consumable fluids of the diagnostic dock 122 and treatment dock 124, such as saline for irrigating the cornea. The diagnostic dock 122 and treatment dock 124 may include structures for electrically connecting to the diagnostic dock 122 and the treatment dock 124 and charging rechargeable batteries in the diagnostic dock 122 and the treatment dock 124.

In some embodiments, the operating environment 100a may include one or more cameras 126. The one or more cameras 126 may have the patient's head 108 in the field of view thereof. The one or more cameras 126 may be used to provide feedback regarding the positions of the diagnostic equipment 118 and treatment equipment 120 relative to the eye of the patient 104.

A controller 128 may be coupled by wires or wirelessly to some or all of the clamp 106 (e.g., actuators for engaging the clamp 106 with the head 108 of the patient 104), the robotic arm 112, end effector 116, diagnostic equipment 118, treatment equipment 120, diagnostic dock 122, treatment dock 124, and camera 126. The controller 128 is a computing device or other electronic device that is programmed or otherwise configured to perform the methods described herein and activate the functionalities ascribed to the components of the operating environment 100a and/or other operating environments described herein below.

The function of the controller 128 may be subject to the control of a surgeon. For example, actions determined by the controller 128 may be invoked only upon receiving approval of a surgeon or may be aborted in response to an instruction from the surgeon. Likewise, the surgeon may direct the controller 128 to invoke the functionalities of the operating environment 100a and/or other operating environments described herein below.

In operation, the robotic arm 112 positions the end effector 116 adjacent the diagnostic equipment 118 located on the diagnostic dock 122. The end effector 116 then secures to the diagnostic equipment 120 by grasping or other selective securement approach. The robotic arm 112 then positions the diagnostic equipment 118 adjacent the head 108 of the patient appropriate for performing the function of the diagnostic equipment 120, such as imaging the eye of the patient 104, measuring refractive error of the eye, measuring IOP of the eye, or performing some other measurement. The robotic arm 112 then returns the diagnostic equipment 118 to the diagnostic dock 122 and the end effector 116 disengages from the diagnostic equipment 118.

The robotic arm 112 then positions the end effector 116 against the treatment equipment 120 located on the treatment dock 124. The end effector 116 then secures to the treatment equipment 120 by grasping or other selective securement approach. The robotic arm 112 then positions the treatment equipment 120 adjacent the head 108 of the patient appropriate for performing the function of the treatment equipment 120, such as performing laser cutting of a flap, laser ablation, creation of a lenticle, or other aspect of a LASIK or SMILE treatment or other ophthalmic treatment.

The operating environment 100a and the other operating environments described herein enable the measurement of the eye and the treatment performed on the eye to be performed close to the same time, e.g., within 30 minutes, within 10 minutes, or a smaller time interval. In this manner, the treatment is less affected by changes in eye geometry due to change in position (sitting vs. supine), drying of the eye, change in temperature, changes due to contact lenses, or other time-varying parameters.

Referring to FIGS. 1B and 1C, in some implementations, equipment utilization may be enhanced using the illustrated operating environment 100b. The operating environment 100b includes two or more treatment areas 130a, 130b separated from one another by a partition 132, such as a curtain, wall defining one or more openings, or other type of partition 132. Each area 130a, 130b may be a room or simply be defined as being the area on one side of the partition 132. Each area 130a, 130b includes a patient support 102 and clamp 106. Each area 130a, 130b may include one or more cameras 126 having the head 108 of a patient clamped by the clamp 106 in the field of view thereof as described above. Each area 130a, 130b includes a robotic arm 112a, 112b, respectively, having the attributes of the robotic arm 112 as described above and including a corresponding end effector 116a, 116b. Each robotic arm 112a, 112b is secured to a corresponding base 114a, 114b, respectively, that is fixed relative to the patient support 102 of the corresponding area 130a, 130b. The bases 114a, 114b may be positioned within the areas 130a, 130b, respectively, i.e., the bases 114a, 114b positioned on the sides of the partition 132 corresponding to the areas 130a, 130b, respectively. In the operating environment 100b, the end effector 116a is permanently or selectively secured to diagnostic equipment 118 and end effector 116b is permanently or selectively secured to treatment equipment 120.

In a first mode of use, robotic arm 112a positions the diagnostic equipment 118 adjacent the head 108 of a patient 104 positioned in the area 130a at a distance and relative position suitable for performing the function of the diagnostic equipment 118. In the first mode of use, the robotic arm 112b positions the treatment equipment 120 adjacent the head 108 of a patient 104 positioned in the area 130b at a distance and relative position suitable for performing the function of the treatment equipment 120.

As shown in FIG. 1C, in a second mode of use, robotic arm 112a passes the diagnostic equipment 118 through the partition 132 to a position adjacent the head 108 of a patient 104 positioned in the area 130b at a distance and relative position suitable for performing the function of the diagnostic equipment 118. In the second mode of use, the robotic arm 112b extends the treatment equipment 120 through the partition 132 and positions the treatment equipment 120 adjacent the head 108 of a patient 104 positioned in the area 130a at a distance and relative position suitable for performing the function of the treatment equipment 120. The partition 132 may be a curtain defining one or more slits or other opening or wall defining one or more openings permitting the robotic arms 112a, 112b to pass therethrough while still preserving patient privacy.

Referring to FIG. 1D, in a third operating environment 100c diagnostic equipment 118 and treatment equipment 120 are mounted to an actuator 134, such as a turntable, rail or channel and corresponding actuator facilitating linear translation, a gantry performing movement in two or more degrees of freedom, or other type of actuator. The actuator 134 may therefore selectively place either the diagnostic equipment 118 or the treatment equipment 120 in a position and orientation to perform the function thereof with respect to an eye 136 of a patient 104. The actuator 134 may possess a range of movement sufficient to move the diagnostic equipment 118 and the treatment equipment 120 into proximity and alignment with both eyes 136 of a patient in order to perform the functions of the diagnostic equipment 118 or the treatment equipment 120 with respect to both eyes 136 of the patient.

In some embodiments, the diagnostic equipment 118 and treatment equipment 120 each include multiple items of equipment. For example, the diagnostic equipment 118 may include a refractometer 118a for measuring refractive error of an eye and an OCT 118b for measuring geometry of the eye, particularly of the cornea. The treatment equipment 120 may include a laser 120a for cutting a flap and a laser 120b for performing ablation in a LASIK procedure. The actuator 134 or a separate actuator may bring a particular item of equipment 118a, 118b, 120a, 120b into proximity and alignment with the eye 136 of a patient required for the function of that item of equipment 118a, 118b, 120a, 120b. Alignment may be performed in combination with one or more cameras, images from which are used to perform eye tracking to align a particular item of equipment 118a, 118b, 120a, 120b with the optical axis of an eye 136 and dock a particular item of equipment 118a, 118b, 120a, 120b the eye 136, which may include suction contact with the eye 136.

A robotic arm 138 may be provided having a base 140 that is fixed relative to the actuator 134 and that performs one or more functions in cooperation with one or both of the diagnostic equipment 118 and the treatment equipment 120. For example, the robotic arm 138 may include an end effector 142 embodied as forceps for grasping and extracting a lenticle or for inserting an artificial lenticle. The end effector 142 may be embodied as other instruments such as a phaco-vit instrument, instrument for placing a shunt or creating an incision for treating glaucoma, instrument for supplying infusion fluid, instrument for providing illumination light, instrument for supplying laser light for retinal attachment, or other type of ophthalmic instrument.

In the operating environments 100a, 100b, the diagnostic equipment 118 may also include a refractometer 118a and an OCT 118b. Likewise, the treatment equipment 120 of the operating environments 100a, 100b may likewise include multiple lasers 120a, 120b. Selective placement of the refractometers 118a and OCT 118b in an appropriate position and alignment with the eye 136 may be accomplished using a robotic arm 112, 112a or a separate actuator 134. Selective placement of each lasers 120a, 120b in position and alignment with the eye 136 may be accomplished using a robotic arm 112, 112b or a separate actuator 134. Alternatively, combining optics may be used to select between the refractometer 118a and OCT 118b and between the multiple lasers 120a, 120b.

The illustrated operating environments 100a, 100b, 100c are exemplary only. For example, in other embodiments, the patient 104 may be moved between positions whereas the diagnostic equipment 118 and treatment equipment 120 are stationary, e.g., stationary other than fine adjustment (e.g., less than 4 cm) to align with the eye 136 of the patient 104. For example, a patient support 102 may be implemented as a patient bed 102 that may be actuated between two or more positions. For example, a surgeon may actuate a foot pedal, joystick, or other interface to invoke actuation of the patient bed between the two or more positions.

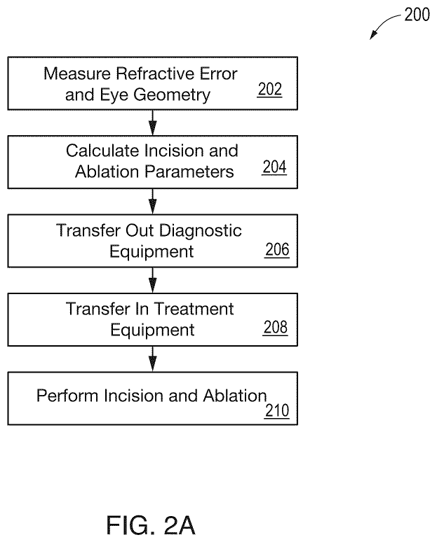

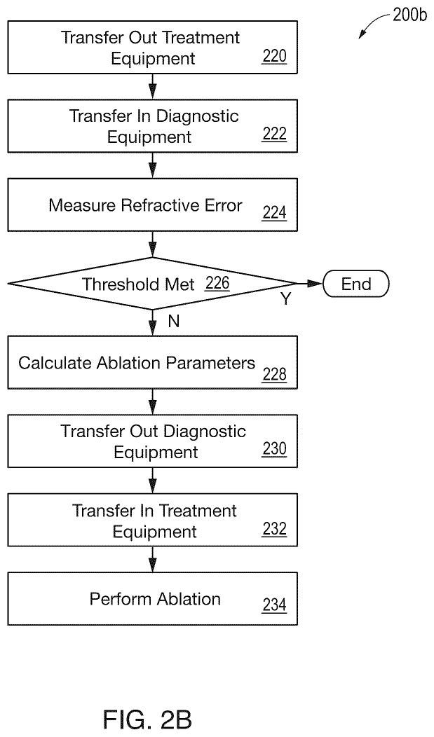

FIGS. 2A and 2B illustrated methods 200a, 200b that may be performed using any of the operating environments 100a, 100b, 100c described above. The methods 200a, 200b refer specifically to performing a LASIK procedure. The methods 200a, 200b may be performed with respect to a patient 104 with the head 108 of the patient being restrained using the clamp 106 continuously during the entire method 200a or method 200b or during performance of both of the methods 200a, 200b.

Referring specifically to FIG. 2A, the method 200a may include measuring, at step 202, the refractive error and eye geometry of a patient's eye using the diagnostic equipment 118. Step 202 may include measuring the refractive error and eye geometry of both eyes of the patient using the diagnostic equipment 118. Step 202 may include performing translations of the diagnostic equipment 118 to bring a refractometer 118a and OCT 118b into proximity and alignment with each eye 136 required to perform the functions of the refractometer 118a and OCT 118b. Alternatively, a beam splitter or other optics may be used to direct light to and from refractometer 118a and the OCT 118b without translation. Geometry of the eye may include the radius of curvature of the cornea 300, thickness of the cornea, or other geometry.

At step 204, the method 200a includes calculating parameters for performing a LASIK procedure based on the measured refractive error and the eye geometry for each eye measured at step 202. The parameters may include parameters for cutting a flap, such as a cutting plane position along the optical axis of the eye. The parameters may include parameters defining ablation of the cornea following cutting of the flap. Calculating the parameters at step 204 may be performed according to any approach for performing a LASIK procedure.

Step 204 may include taking into account prior measurements of refractive error and/or eye geometry, such as those calculated on a different occasion one or more days in the past. For example, step 202 may include modifying parameters calculated from the prior measurements based on changes in refractive error and/or eye geometry from step 202. Step 204 may also take into account time elapsed between performing step 202 and when ablation and incision (see description of step 210 below) are performed to account for drying of the eye or other time varying attributes of the eye 136.

At step 206, the diagnostic equipment 118 is transferred out and, at step 208, the treatment equipment 120 is transferred in. In particular, as used herein, transferring in equipment shall be understood as transferring into a region in front of an eye 136 at which the equipment can perform the function thereof, whether that of the diagnostic equipment 118 or of the treatment equipment 120. Transferring in may be image guided, i.e., images from the camera 126 or on an item of equipment may be used to perform eye tracking and align the item of equipment with the eye and at the correct distance from the eye or in contact with the eye. For example, some types of treatment laser may establish a suction contact with the eye being treated thereby.

Transferring out equipment includes transferring the equipment out of the region such that a different item of equipment may be positioned within the region and perform the function of the different item of equipment, whether that of the diagnostic equipment 118 or treatment equipment 120. Transferring in and transferring out may be performed as described above with respect to any of the operating environments 100a, 100b, 100c. Between transferring out of one item of equipment and transferring in of another item of equipment, one or more other steps may be performed, such as insertion of a speculum.

The method 200 may then include performing, at step 210, incision and ablation according to the parameters calculated at step 204 using the treatment equipment 120. Performing incision and ablation may include performing eye tracking (e.g., tracking movement of the iris) or immobilizing the eye 136. Where eye tracking is performed, treatment lasers of the treatment equipment 120 may be actuated to compensate for movement of the eye 136.

In some embodiments, following performing incision, the method 200a may include transferring out the treatment equipment 120 and transferring in the diagnostic equipment 118 in order to measure the geometry of the eye following incision. Ablation parameters may then be updated or recalculated based on the actual geometry of the eye 136 following incision, thereby accounting for any error in the actual incision and the incision parameters calculated at step 204. The diagnostic equipment 118 may then be transferred out and the treatment equipment 120 transferred in in order to perform ablation according to the ablation parameters. Ablation parameters may be modified based on the amount of time that has passed from the time that the incision was made.

Referring specifically to FIG. 2B, the method 200b may be performed following performance of the method 200a. At step 220, the treatment equipment 120 is transferred out and, at step 222, the diagnostic equipment 118 is transferred in.

The method 200b may include measuring, at step 224 refractive error of one or both eyes of the patient. Step 224 may be preceded by folding a flap over the ablated area of the cornea from the method 200a. The method 200b may include evaluating, at step 226, refractive error for each eye measured at step 224 with respect to a threshold. Step 226 may include adjusting the refractive error and comparing the adjusted refractive error to the threshold. The adjustment may account for changes in refractive error that occur after the flap has healed. The adjustment may therefore be the obtained by analyzing measurements of intra-operative and post-healing refractive errors for previous LASIK treatments. For example, a machine learning model may be trained to relate pre-healing values to a post-healing refractive error measurement, the pre-healing values including some or all of pre-operative refractive error, pre-operative eye geometry, incision and ablation parameters (describing actual incision and ablation and/or as calculated), and intra-operative refractive error (e.g., measured after ablation but before healing).

If the refractive error is found to meet the threshold at step 226, the method 200b may end. If the refractive error is not found to meet the threshold at step 226, then the method 200b may include calculating, at step 228, ablation parameters in order to reduce the refractive error. The ablation parameters may be calculated according to any approach known in the art for performing LASIK treatments.

The method 200b may include transferring out the diagnostic equipment 118 at step 230 and transferring in the treatment equipment 120 at step 232. At step 234, ablation is again performed according to the parameters calculated at step 228. Step 234 may be preceded by folding back the flap of the cornea created by the incision. Steps 224-234 may be performed any number of times until the threshold of step 226 is found to be met.

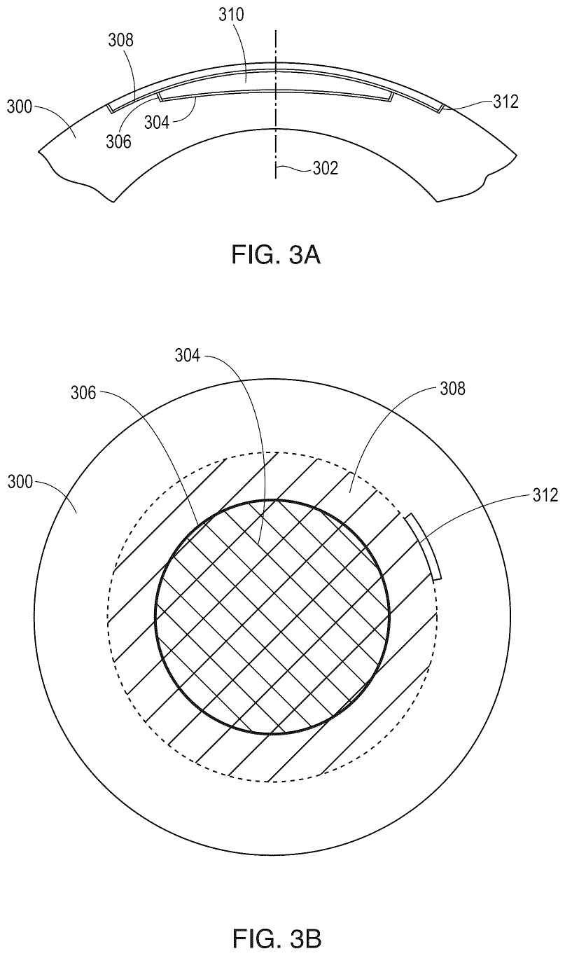

FIGS. 3A and 3B illustrate a SMILE treatment. In a SMILE treatment, one or more lasers are used to make a series of cuts within the cornea 300. The cuts are described below with reference to the optical axis 302 of the cornea 300. The cuts described below may be made in the order described or some other order. The cuts may be made according to any approach known in the art for performing SMILE treatments.

A lenticle cut 304 is the deepest cut and may be a circular area perpendicular to and centered on the optical axis 302. As is apparent in FIG. 3A, a portion of the cornea 300 extends around the lenticle cut 304 in a plane parallel to the lenticle cut 304. Lenticle side cut 306 extend around the perimeter of the lenticle cut 304 parallel to the optical axis 302. As shown in FIG. 3, the lenticle side cut 306 does not extend completely to the outer surface of the cornea 300.

A cap cut 308 is offset above the lenticle cut 304. As shown in FIG. 3A, the cap cut 308 may be at a uniform depth below the surface of the cornea 300. The cap cut 308 extends outwardly from the lenticle cut 304 and the lenticle side cut 306 ends at the cap cut 308. The region between the lenticle cut 304, lenticle side cut 306, and cap cut 308 is the lenticle 310, which is detached from the rest of the cornea 300. The lenticle 310 is removed through an incision 312 extending from the outer surface of the cornea 300 to the cap cut 308.

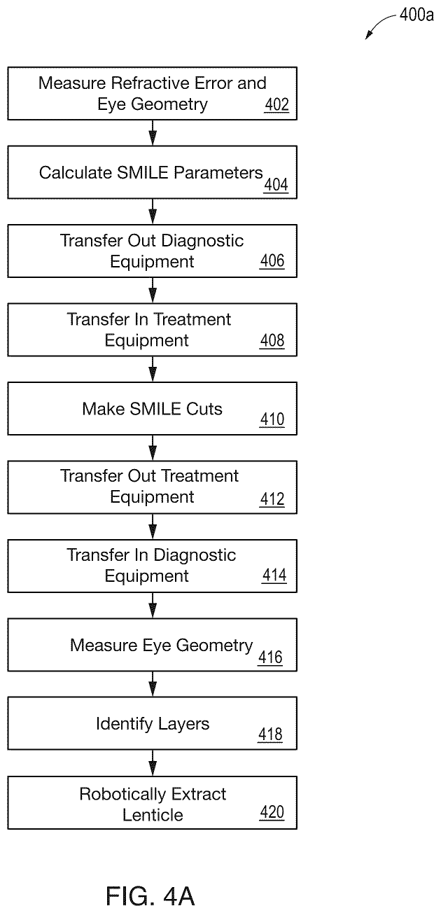

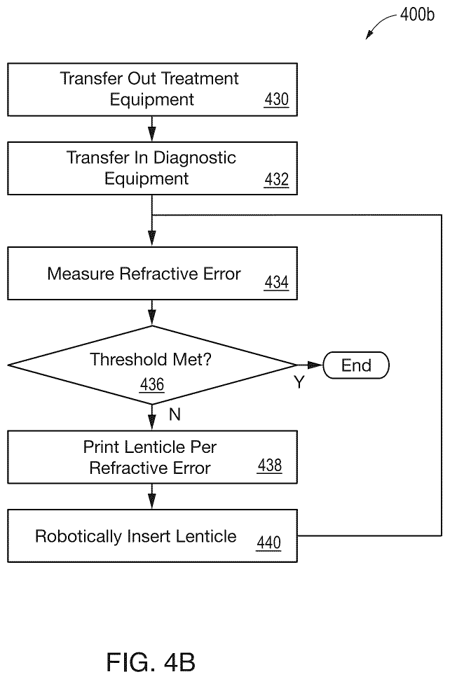

FIGS. 4A and 4B illustrate methods 400a, 400b that may be performed using any of the operating environments 100a, 100b, 100c. The methods 400a, 400b may be performed with the head 108 of the patient being restrained using the clamp 106 continuously during the entire method 400a or 400b or during performance of both of the methods 400a, 400b.

The method 400a includes measuring refractive error and geometry of the eye 136 at step 402 and using the diagnostic equipment 118. For example, refractive error may be measured using a refractometer 118a and geometry of the eye may be measured using an OCT 118b. The method 400a includes calculating, at step 404, SMILE parameters. The SMILE parameters may include a depth and diameter of the lenticle cut 304, a depth of the cap cut 308, a location of the incision 312, or other parameters. The SMILE parameters may be calculated according to any approach known in the art.

The method 400a includes transferring out the diagnostic equipment 118 at step 406 and transferring in the treatment equipment 120 at step 408. The method 400a then includes making SMILE cuts at step 410 according to the smile parameters calculated at step 404. Step 410 may include making the lenticle cut 304, lenticle side cut 306, cap cut 308, and incision 312 as specified in the SMILE parameters. The method 400a may then include transferring out the treatment equipment 120 at step 412 and transferring in the diagnostic equipment 118 at step 414. The geometry of the eye 136 is then measured at step 416, such as with an OCT. The measurement of step 416 may image the layers created by the cuts from step 410. The layers represented in the measurements of step 416 may then be identified at step 418. The lenticle 310 is then extracted at step 420, such as using the robotic arm 138. For example, steps 416 and 418 may be performed repeatedly during step 420 to provide feedback for guiding the robotic arm 138.

At step 420, the controller 128 may use the identified location of the lenticle 310 and incision 312, and possibly the end effector 142 of the robotic arm 138 itself, to control the robotic arm 138 to insert into the incision 312, grasp the lenticle 310, and withdraw the lenticle from the incision 312. For example, steps 416 and 418 may be used to provide feedback to ensure that the end effector 142 is aligned with the incision both parallel to and perpendicular to the optical axis, to align the end effector with the cap cut 308, lenticle cut 304, or lenticle 310 itself, or perform other type of alignment.

The end effector 142 may incorporate a torque sensor that measures torque exerted through the end effector in order to provide feedback to the controller 128, e.g., determine whether excessive torque is required to separate layers (e.g., between cap cut 308 and lenticle cut 304) such that lenticle extraction to be aborted. For example, the end effector 142 may include probes with corresponding one or more torque sensors that are used separate the layers and separate forceps for grasping the lenticle 310.

Referring to FIG. 4B, the method 400b may be performed after performing the method 400a. The method 400b may include transferring out the treatment equipment 120 at step 430 and transferring in the diagnostic equipment at step 432. The method 400b may include measuring the refractive error of the eye 136 at step 434. The refractive error of the eye 136 may then be evaluated with respect to a threshold at step 436. If the refractive error is below the threshold, the method 400b may end. Step 436 may include adjusting the refractive error to account for change in refractive error due to healing of the cornea 300 and comparing the adjusted refractive error to the threshold. The adjustment may therefore be the obtained by analyzing measurements of intra-operative and post-healing refractive errors for previous SMILE treatments. For example, a machine learning model may be trained to relate pre-healing values to a post-healing refractive error measurement, the pre-healing values including some or all of pre-operative refractive error, pre-operative eye geometry, SMILE parameters, and intra-operative refractive error (e.g., measured after lenticle removal but before healing).

If the refractive error is found to be below the threshold, the method 400b may end. If the refractive error is found to be above the threshold, the method 400b may include creating a replacement lenticle based on the refractive error. For example, an artificial lenticle may be created at step 438. The artificial lenticle may be created using three-dimensional printing of a biocompatible transparent material. The dimensions of the artificial lenticle may be selected based on the measured refractive error from step 434 and one or more other values such as the pre-operative refractive error, pre-operative eye geometry, post-cut eye geometry (e.g., after lenticle extraction of step 420), and refractive error from step 434.

The artificial may then be inserted through the incision 312 into the cavity formerly occupied by the lenticle 310 at step 440. Step 440 may be performed using the robotic arm 138. For example, the end effector 142 may grasp the artificial lenticle and insert the artificial lenticle through the incision 312. Following insertion of the artificial lenticle, the method 400b may end or may repeat starting at step 434. If repeated, the method 400b may include removing a previously inserted artificial lenticle prior to inserting a new artificial lenticle.

The methods 200a, 200b and the methods 400a, 400b are exemplary only. The operating environments 100a, 100b, 100c may be used to perform other refractive correction surgeries, such as PRK. In some embodiments, measurements of refractive error and eye geometry obtained using the diagnostic equipment may be used to select from among multiple refractive correction surgeries, such as LASIK, SMILE, PRK, ICL placement, etc.

### ADDITIONAL CONSIDERATIONS

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A system for performing ophthalmic surgery, the system comprising:
an actuator;
one or more items of diagnostic equipment configured to perform ophthalmic measurement;
one or more items of treatment equipment configured to facilitate performance of an ophthalmic treatment with respect to an eye of a patient; and
a controller coupled to the actuator and configured to cause the actuator to transfer the one or more items of diagnostic equipment and the one or more items of treatment equipment into and out of a region in front of an eye of the patient.

2. The system of claim 1, wherein the actuator is a robotic arm.

3. The system of claim 2, wherein the controller is configured to cause the robotic arm to:
secure to the one or more items of treatment equipment;
release the one or more items of treatment equipment;
secure to the one or more items of diagnostic equipment; and
release the one or more items of diagnostic equipment.

4. The system of claim 3, further comprising a diagnostic dock and a treatment dock, the controller configured to release the one or more items of diagnostic equipment onto the diagnostic dock and release the one or more items of treatment equipment onto the treatment dock.

5. The system of claim 1, wherein the actuator is a first robotic arm secured to the one or more items of diagnostic equipment and a second robotic arm secured to the one or more items of treatment equipment.

6. The system of claim 5, further comprising a partition positioned between a first base of the first robotic arm and a second base of the second robotic arm.

7. The system of claim 1, wherein the one or more items of diagnostic equipment includes an optical coherence tomography (OCT) imaging device.

8. The system of claim 1, wherein the one or more items of diagnostic equipment includes a refractometer.

9. The system of claim 1, wherein the one or more items of diagnostic equipment includes an optical coherence tomography (OCT) imaging device and a refractometer.

10. The system of claim 1, wherein the one or more items of treatment equipment include one or more treatment lasers.

11. The system of claim 10, wherein the one or more treatment lasers are configured to perform as such as laser-assisted in-situ keratomileusis (LASIK) treatment.

12. The system of claim 10, wherein the one or more treatment lasers are configured to perform a small incision lenticular extraction (SMILE) treatment.

13. The system of claim 1, further comprising a robotic arm including an end effector, the controller configured to cause the robotic arm and end effector to remove a lenticle from the eye of the patient.

14. A method comprising:
positioning a head of a patient in a restraint;
while the head of the patient remains in the restraint:
(a) activating, by a controller, one or more actuators to move one or more items of diagnostic equipment configured to perform ophthalmic measurement into a region in front of an eye of the patient;
(b) activating, by the controller, the one or more items of diagnostic equipment to obtain measurement data;
(c) activating, by the controller, the one or more actuators to move the one or more items of diagnostic equipment out of the region;
(d) activating, by the controller, the one or more actuators to move one or more items of treatment equipment configured to perform an ophthalmic treatment into the region; and
(e) activating, by the controller, the one or more items of treatment equipment to perform the ophthalmic treatment according to the measurement data.

15. The method of claim 14, wherein the measurement data is a refractive error of the eye.

16. The method of claim 15, wherein the ophthalmic treatment is a laser-assisted in-situ keratomileusis (LASIK) treatment.

17. The method of claim 15, wherein the ophthalmic treatment is a small incision lenticular extraction (SMILE) treatment.

18. The method of claim 15, wherein the refractive error is a first refractive error, the method further comprising, following performing (e):
(f) activating, by a controller, the one or more actuators to move the one or more items of treatment equipment out of the region;
(g) activating, by a controller, the one or more actuators to move the one or more items of diagnostic equipment into the region;
(h) activating, by the controller, the one or more items of treatment equipment to measure a second refractive error of the eye of the patient;
(i) activating, by the controller, the one or more actuators to move the one or more items of diagnostic equipment out of the region;
(j) activating, by the controller, the one or more actuators to move one or more items of treatment equipment into the region; and
(e) activating, by the controller, the one or more items of treatment equipment to perform the ophthalmic treatment according to the second refractive error.

19. The method of claim 14, wherein the one or more actuators are a single robotic arm.

20. The method of claim 14, wherein the one or more actuators comprise:
a first robotic arm coupled to the one or more items of diagnostic equipment;
a second robotic arm coupled to the one or more items of treatment equipment.
